(19) United States
(12) Patent Application Publication
(10) Pub. No.: US 2024/0112810 A1
(43) Pub. Date: Apr. 4, 2024

(54) SYSTEM FOR PERFORMING ARABIC APHASIA TESTS ON A PATIENT

(71) Applicant: SALT LabSystem Limited, Sheffield (GB)

(72) Inventor: Tariq Khwaileh, Sheffield (GB)

(21) Appl. No.: 17/936,966

(22) Filed: Sep. 30, 2022

(51) Int. Cl. G16H 50/30; A61B 5/00; G16H 50/20

(52) U.S. Cl. CPC G16H 50/30; A61B 5/4088; G16H 50/20

(57) ABSTRACT

The subject-matter of the present disclosure relates to a system for performing Arabic aphasia tests on a patient. The system comprises: a testing portal device, the testing portal device in communication with a patient device and a tester device, the testing portal device configured to: receive, from the patient device, a plurality of tests presented to the patient by the patient device during testing, and a plurality of corresponding answers input to the patient device by the patient; receive, from the tester device, a plurality of marks, each mark corresponding to an answer input to the patient device indicating that the patient answered the test correctly; and generate a report indicating results of the Arabic aphasia tests based on the plurality of tests, the plurality of answers, and the plurality of marks

### FIELD

The subject-matter of the present disclosure relates to the field of Arabic aphasia testing. More specifically, the subject-matter of present disclosure relates to a system for performing Arabic aphasia tests on a patient, a computer-implemented method of performing Arabic aphasia tests on a patient, and a non-transitory computer-readable medium.

### BACKGROUND

Speech language therapy methods testing for aphasia are well known. Such tests are typically carried out in person by a tester, or therapist. Results of the tests are captured manually by the tester using notes taken during the test, often on paper, using stopwatches, using voice recorders and scoring sheets. Paper based testing is an administrative burden, and in-person testing is a logistical burden especially for disabled people where a tester may need to travel to visit patients in different areas.

The subject-matter of the present disclosure aims to address such issues and improve on the prior art.

### SUMMARY

According to an aspect of the present disclosure, there is provided a system for performing Arabic aphasia tests on a patient. The system comprising: a testing portal device, the testing portal device in communication with a patient device and a tester device, the testing portal device configured to: receive, from the patient device, a plurality of tests presented to the patient by the patient device during testing, and a plurality of corresponding answers input to the patient device by the patient; receive, from the tester device, a plurality of marks, each mark corresponding to an answer input to the patient device indicating that the patient answered the test correctly; and generate a report indicating results of the Arabic aphasia tests based on the plurality of tests, the plurality of answers, and the plurality of marks.

In this way, the administrative burden is relieved from the tester because the report is generated automatically by the tester portal device rather than relying on retrospective compiling of the report by the tester. In addition, the overall user experience, from the perspectives of both the tester and the patient is improved.

In an embodiment, the system may be a speech and/or language therapy system.

In an embodiment, the testing portal device is configured to generate an overall aphasia quotient by: calculating an aphasia quotient using AQ=(S/N)×100, where AQ is the aphasia quotient, S is a score calculated by counting a number of marks received for a subset of the plurality of tests, and N is a total number of tests within the subset of the plurality of tests; and calculating the overall aphasia quotient by calculating a mean value using respective aphasia quotients from a plurality of subsets of the plurality of tests, wherein each subset of the plurality of tests may correspond to a category of test, wherein the report may include the overall aphasia quotient and the aphasia quotient for each category of test.

In an embodiment, the testing portal device may be configured to classify automatically an aphasia subtype by comparing each aphasia quotient of a respective category of test with a respective threshold, and to classify the aphasia subtype depending on whether one or more of the aphasia quotients is below the respective threshold, wherein the report may include the automatically classified aphasia subtype.

In an embodiment, the aphasia subtype may be selected from a list including anomic aphasia, conduction aphasia, transcortical motor aphasia, Wernicke's aphasia, transcortical sensory aphasia, Broca's aphasia, isolation aphasia, and global aphasia.

In an embodiment, the category of tests may include Arabic apraxia screening, Arabic dysarthria screening, Arabic quick aphasia screening, Arabic comprehensive aphasia testing, Arabic naming testing, and Arabic agrammatism testing.

In an embodiment, the system may further comprise the patient device, wherein the patient device may be in communication with the tester device and may be configured to: output a stimulus to the patient to present each of the plurality of tests; and receive an input from the patient, wherein the input includes a response to the respective test.

In an embodiment, the stimulus may be a stimulus selected from a list including an auditory stimulus and a visual stimulus.

In an embodiment, when the stimulus is an auditory stimulus, the auditory stimulus may include an auditory stimulus comprises spoken Arabic.

In an embodiment, when the stimulus is a visual stimulus, the visual stimulus may include a visual stimulus selected from a list including Arabic text and an image.

In an embodiment, the input from the patient may be an input selected from a list including a tactile input, and an auditory input.

In an embodiment, the patient device may be configured to measure a response time, wherein the response time may be a time between a first time point when display of a test of the plurality of tests is commenced, and a second time point when a patient has finished inputted the corresponding answer.

In an embodiment, the system may further comprise the tester device, wherein the tester device may be in communication with the patient device and may be configured to: output, to the tester, a test of the plurality of tests that is, in real-time, being output to the patient by the patient device; output, to the tester, the answers that are, in real-time, being input by the patient to the patient device; receive an input from a tester to control the plurality of tests being presented by the patient device; and controlling the plurality of tests being presented by the patient device based on the received input from the tester.

In an embodiment, the controlling the plurality of tests being presented by the patient device may comprise a controlling action selected from a list including skipping a test, jumping a test, interrupting a test, terminating a test, and re-ordering the plurality of tests.

In an embodiment, the tester device may be configured to: receive a mark from the tester, the mark indicating that an answer to a corresponding test is correct; and send the mark to the testing portal device together with the corresponding question.

In an embodiment, the tester device may be configured to display a response time, wherein the response time may include a timer starting at a first time point, the first time point corresponding to a time when the test starts to be presented on the patient device, and wherein the tester device may be configured to receive an input from the tester to stop the timer.

According to an aspect of the present disclosure, there is provided a computer-implemented method of performing Arabic aphasia tests on a patient, the method comprising: receiving, by a testing portal device from a patient device, a plurality of tests presented to a patient by the patient device during testing, and a plurality of corresponding answers input to the patient device by the patient; receiving, by the testing portal device from a tester device, a number of correct answers corresponding to the answers input to the patient device; and generating, by the testing portal device, a report based on the plurality of tests, the plurality of answers, and the number of correct answers.

In this way, the administrative burden is relieved from the tester because the report is generated automatically by the tester portal device rather than relying on retrospective compiling of the report by the tester. In addition, the overall user experience, from the perspectives of both the tester and the patient is improved.

In an embodiment, the generating the report may comprise generating an overall aphasia quotient by: calculating, by the testing portal device, an aphasia quotient using a formula AQ=(S/N)×100, where AQ is the aphasia quotient, S is a score calculated by counting the number of marks awarded for a subset of the plurality of tests, and N is a total number of tests within the subset of the plurality of tests; and calculating, by the testing portal device, the overall aphasia quotient by calculating a mean value using respective aphasia quotients from a plurality of subsets of the plurality of tests, wherein each subset corresponds to a category of test, wherein the report includes the overall aphasia quotient and the aphasia quotient for each category of test.

In an embodiment, the method may further comprise classifying automatically an aphasia subtype by comparing each aphasia quotient of a respective category of test with a respective threshold, and classifying the aphasia subtype depending on whether one or more of the aphasia quotients is below the respective threshold, wherein the report includes the automatically classified aphasia subtype.

In an embodiment, the method may further comprise: outputting, by the patient device, a stimulus to the patient to present each of the plurality of tests; and receiving, by the patient device, an input from the patient, wherein the input includes a response to the respective test.

In an embodiment, the method may further comprise generating an overall aphasia quotient by: calculating an aphasia quotient using AQ=(S/N)×100, where AQ is the aphasia quotient, S is a score calculated by counting a number of marks received for a subset of the plurality of tests, and N is a total number of tests within the subset of the plurality of tests; and calculating the overall aphasia quotient by calculating a mean value using respective aphasia quotients from a plurality of subsets of the plurality of tests, wherein each subset of the plurality of tests may correspond to a category of test, wherein the report may include the overall aphasia quotient and the aphasia quotient for each category of test.

In an embodiment, the method may further comprise classifying automatically, by the tester portal device, an aphasia subtype by comparing each aphasia quotient of a respective category of test with a respective threshold, and classifying, by the tester portal device, the aphasia subtype depending on whether one or more of the aphasia quotients is below the respective threshold, wherein the report may include the automatically classified aphasia subtype.

In an embodiment, the aphasia subtype may be selected from a list including anomic aphasia, conduction aphasia, transcortical motor aphasia, Wernicke's aphasia, transcortical sensory aphasia, Broca's aphasia, isolation aphasia, and global aphasia.

In an embodiment, the category of tests may include Arabic apraxia screening, Arabic dysarthria screening, Arabic quick aphasia screening, Arabic comprehensive aphasia testing, Arabic naming testing, and Arabic agrammatism testing.

In an embodiment, the method may further comprise outputting, by a patient device, a stimulus to the patient to present each of the plurality of tests; and receiving, by the patient device, an input from the patient, wherein the input may include a response, or answer, to the respective test.

In an embodiment, the stimulus may be a stimulus selected from a list including an auditory stimulus and a visual stimulus.

In an embodiment, when the stimulus is an auditory stimulus, the auditory stimulus may include an auditory stimulus comprises spoken Arabic.

In an embodiment, when the stimulus is a visual stimulus, the visual stimulus may include a visual stimulus selected from a list including Arabic text and an image.

In an embodiment, the input from the patient may be an input selected from a list including a tactile input, and an auditory, or phonetic, input.

In an embodiment, the method may further comprise measuring, by the patient device, a response time, wherein the response time may be a time between a first time point when display of a test of the plurality of tests is commenced, and a second time point when a patient has finished inputted the corresponding answer.

In an embodiment, the method may further comprise outputting, by the tester device, to the tester, a test of the plurality of tests that is, in real-time, being output to the patient by the patient device; outputting, by the tester device, to the tester, the answers that are, in real-time, being input by the patient to the patient device; receiving, by the tester device, an input from a tester to control the plurality of tests being presented by the patient device; and controlling, by the tester device, the plurality of tests being presented by the patient device based on the received input from the tester.

In an embodiment, the controlling the plurality of tests being presented by the patient device may comprise a controlling action selected from a list including skipping a test, jumping a test, interrupting a test, terminating a test, and re-ordering the plurality of tests.

In an embodiment, the method may further comprise receiving, by the tester device, a mark from the tester, the mark indicating that an answer to a corresponding test is correct; and sending, by the tester device, the mark to the testing portal device together with the corresponding question.

In an embodiment, the method may further comprise displaying, by the tester device, a response time, wherein the response time may include a timer starting at a first time point, the first time point corresponding to a time at which the test presented on the patient device commences, and receiving, by the tester device, an input from the tester to stop the timer.

According to an aspect of the present disclosure, there is provided a non-transitory computer-readable medium including instructions stored thereon that when executed by a processor, cause the processor to perform the method of claim 16.

### BRIEF DESCRIPTION OF DRAWINGS

The embodiments described herein are described with reference to the accompanying figures, in which:

FIG. 1 shows a flow chart representing human language processing of a single word;

FIG. 2 shows a flow chart representing human language processing for comprehension of a sentence;

FIG. 3 shows a flow chart representing human language processing for production of a sentence;

FIG. 4 shows a flow chart representing human language processing for production of a single word from visual stimuli;

FIG. 5 shows a flow chart representing human language processing for production of a single word, or a single non-word, from text;

FIG. 6 shows a flow chart representing human language processing for repeating a single word or non-word;

FIG. 7 shows a block diagram of a language therapy system according to one or more embodiments for testing the language speech processes governed by flow charts in FIGS. 1 to 6;

FIG. 8 shows a block diagram of the language therapy system from FIG. 7 detailing different tests carried out by the speech therapy system;

FIG. 9 shows a screen shot of a patient device from FIG. 7 displaying a test being carried out on a patient;

FIG. 10 shows a screen shot of a tester device from FIG. 7 displaying the test being displayed on the patient device in FIG. 9;

FIG. 11 shows a screen shot similar to the screen shot of FIG. 10 of the tester device from FIG. 7 displaying another test being displayed on the patient device in FIG. 9;

FIG. 12 shows screen shot of a tester device from FIG. 7 displaying a test selection menu;

FIG. 13 shows a screen shot of a tester device from FIG. 7 displaying a score input menu;

FIG. 14 shows a screen shot of a testing portal device from FIG. 7 displaying a report generated for AAT;

FIG. 15 shows a similar view as FIG. 14 of a screen shot of a testing portal device from FIG. 7 displaying a report generated for ACAT; and

FIG. 16 shows a flow chart of a computer-implemented method according to one or more embodiments.

### DESCRIPTION OF EMBODIMENTS

The embodiments described herein are embodied as sets of instructions stored as electronic data in one or more storage media. Specifically, the instructions may be provided on a transitory or non-transitory computer-readable media. When executed by the processor, the processor is configured to perform the various methods described in the following embodiments. In this way, the methods may be computer-implemented methods.

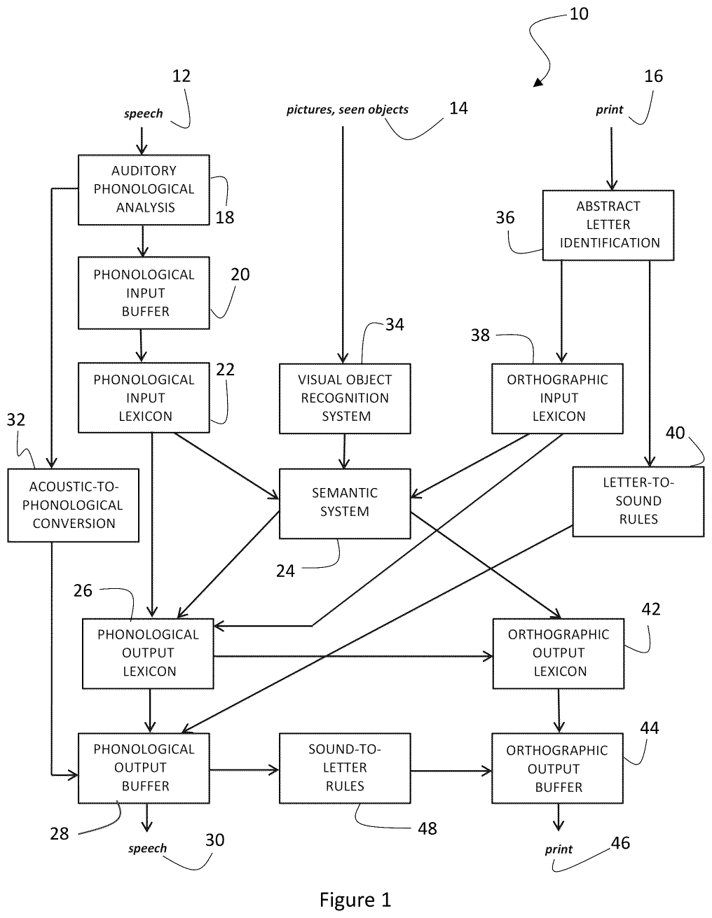

FIGS. 1 to 6 shows flow charts showing various human language processes governing different types of language production and comprehension. Such processes are known.

FIG. 1 shows a flow chart 10 governing human language comprehension and production of a single word.

With reference to FIG. 1, a human receives one or more of three types of stimulus. A first stimulus 12 is hearing a sound, e.g. speech, a second stimulus 14 is viewing an image or an object, and a third stimulus 16 is reading text.

At step 18, a sound, or word, heard by a person is decomposed. This is known as auditory phonological analysis. At step 20, the sound heard by the person is stored in a buffer. This is known as phonological input buffer. At step 22, the stored sound is retrieved and compared to a lexicon of sounds in the human memory to determine if the person is familiar with that sound. This is known as phonological input lexicon. At step 24, the person comprehends the sound by assigning a definition to the term. This is known as the semantic system. At step 26, the person determines if they are familiar with how to articulate that word. This is known as phonological output lexicon. If the semantic system inputs to the phonological lexicon, the person is effectively determining if they are aware of how to pronounce a word they know. If the phonological lexicon receives an input from the phonological input lexicon, the person is effectively determining if they can articulate the word they have just heard, even though they do not comprehend what that word means, e.g. it is a made up word or a real word for which the person does not know the definition. At step 28, the person stores the word to be spoken, which is called the phonological output buffer. At step 30, the person speaks the word from the phonological output buffer and articulates the word. Step 32 covers acoustic-to-phonological conversion, where the person has not even recognised the word but is able to repeat the sounds they have heard.

When a person observes an image or an object, at step 34, their visual object recognition system determines if they recognize the object. If they do recognize the object, the semantic system at step 34 assigns a meaning to the object or image.

When the person reads printed text at 16, at 36, they identify each letter from the text. This is known as abstract letter identification. If the person recognises the letter, they determine if they recognise a word made up of the letters at 38. This is known as orthographic input lexicon. If the user recognises a word from the text, the process proceeds to the semantic system 24. If they do not recognise a word from the text, they are still able to pronounce the word by proceeding to the phonological output lexicon 26 by-passing the semantic system 24.

At 40, if the person does not recognise a word from the text, they are able to apply letter-to-sound rules to determine a pronunciation for the word they have read. The letter-to-sound rules 40 are output to the phonological output buffer 28, where the word is stored before being spoken at 30.

At 42, the person determines if they know how to write a word that either the semantic system 24 or the phonological output lexicon 26 inputs thereto. This is called the orthographic output lexicon. If they are able to write the word, the orthographic output buffer 44 stores the word for writing, e.g. as part of a sentence. The person ultimately writes the word at 46.

At 48, the person is able to convert a word that is stored for speaking at 28 to a word to be written at 44, by using sound-to-letter rules.

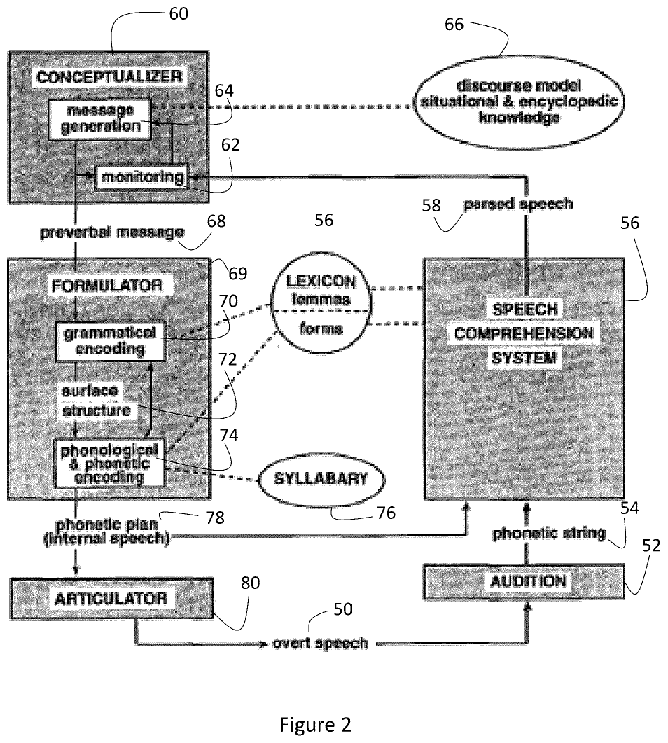

With reference to FIG. 2, a person's internal system for comprehension of a sentence is shown in the form of a flow chart.

At step 50, the person hears speech 52. This is called audition. The output from audition is a phonetic string 54. From the heard speech, the person determines if they recognized the words in the speech as part of the speech comprehension system 56. The output of the speech comprehension system 56 is parsed speech 58.

The parsed speech 58 is input to the conceptualizer 60, where the speech is monitored at 62 and a message is determined at 64 using discourse model situational & encyclopedic knowledge 66. The message is the response to the speech that the person has formulated.

The output from the conceptualizer 60 is a preverbal sentence 68, which is input to a formulator 69. Verbs positions and word order is applied at 70, which is called grammatical encoding. Surface structure is applied at 72, and the sound for producing the sentence is created at 74, also called phonological & phonetic encoding, using syllabary 76 as another input thereto.

The output of the formulator 69 is a phonetic plan 78, which is effectively the internal speech within the mind of a person. The phonetic plan 78 is then output to the articulator 80, where the person articulates the speech out loud.

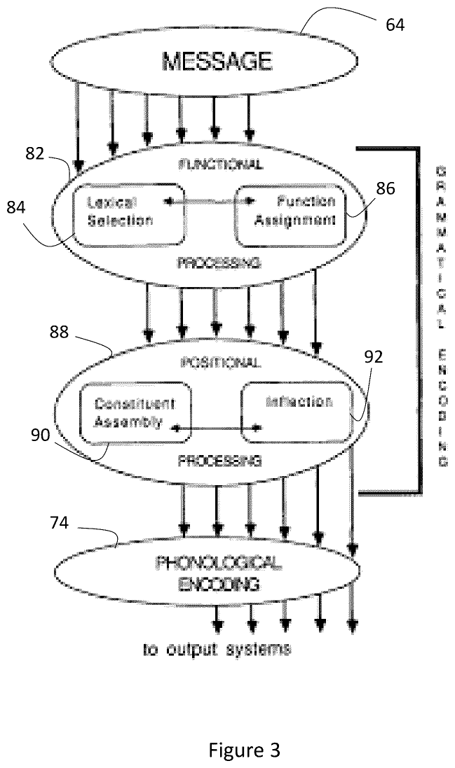

FIG. 3 shows a flow chart for sentence processing (production). FIG. 3 is another way of representing the formulator 69 from FIG. 2.

The message 64 is input to a functional processing step 82, where a lexical selection 84 and a function assignment 86 are applied. The functional processing effectively amounts to what the words represent semantically.

The next step is 88, which is a positional processing step. In this step, constituent assembly 90 is applied, which effectively amounts to ordering the words created at step 82. Any infractions are corrected at 92. Next phonological encoding 74 takes place as per FIG. 2.

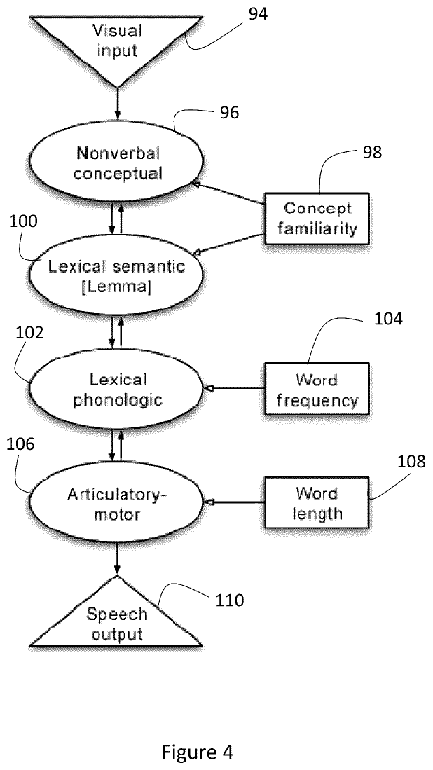

FIG. 4 shows a flow chart representing how a person produces a single word from visual stimuli, e.g. an image of an object.

At 94 the person observes the visual stimuli. At 96, the person determines if there is an object in the image and compares the object to their memory to determine if they are familiar with the object, at step 98. At 100, the person assigns a meaning to the object if they are aware what the object is. This is known as lexical semantic.

Next, at 102, the person determines if they know how to pronounce the name of the object, and calls on the frequency of having understood that word before, at step 104.

At 106, the person determines a pronunciation for the word, and calls on a known word length from memory at 108. At 110, the person outputs the word as speech.

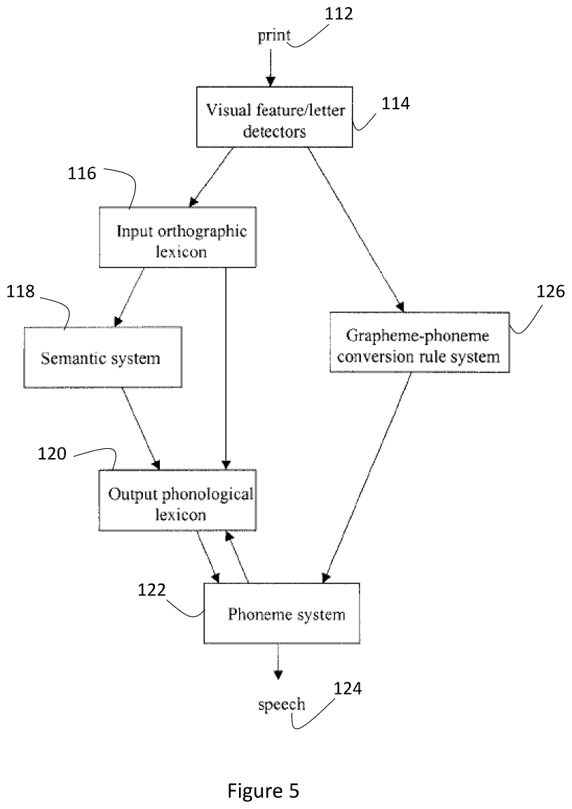

FIG. 5 is a similar flow chart to FIG. 4 but of a person reading words from text rather than viewing objects in an image.

At 112, the person reads the text. At 114, the person detects individual letters in the text. At 116, the person determines if they recognize a word made up of the letters. This is known as the input orthographic lexicon. If they do, at 118, the semantic system provides comprehension to the word. The output of the comprehended word is the output phonological lexicon where the person determines if they know how to pronounce the word, at 120. An input to the output phonological lexicon also comes from the input orthographic lexicon 116 if the person does not recognise the word. Such cases where this can happen is where the word is a real word but the person does not know it. At 122, the person determines an articulation to pronounce the text. This is called a phoneme system. The output of the phoneme system 122 is for the person to verbally say the text at 124. If the person does not recognize the word, e.g. if it is a made up word, at 126, the person applies graphene-phoneme conversion rule system, which is input directly to the phoneme system.

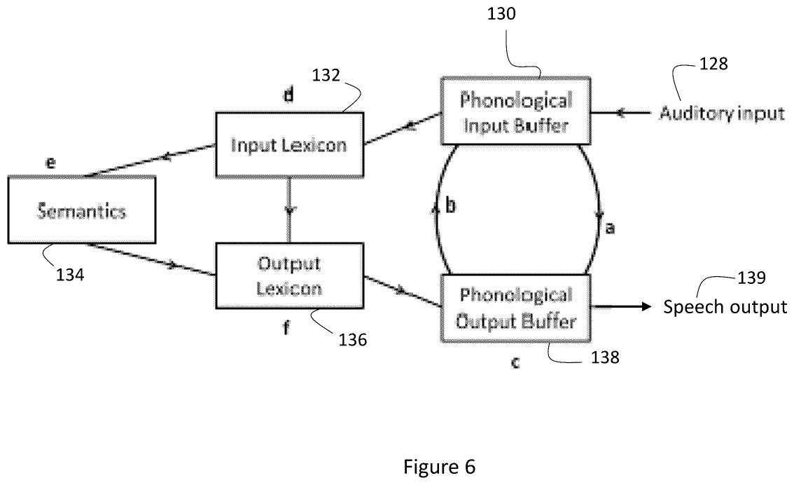

FIG. 6 is a flow chart showing how a person repeats a word or a non-word. A word or non-word is heard at 128, and is input to the phonological input buffer 130. An input lexicon 132 determines is the user recognizes the word or not. If the person does recognise the word, the semantics 134 applies a meaning to the word, which is then applied to an output lexicon where the person determines if they are able to pronounce the word. If the input lexicon 132 determines that the person does not recognise the word, e.g. is it a word but they do not know it's meaning, the output lexicon is then triggered where the user determines a pronunciation for the word. At 136, the phonological output buffer receives the pronunciation for outputting. If the person does not believe the word is a word, instead of the phonological input buffer passing to the input lexicon, the non-word may pass directly to the phonological output buffer 138 where the person can literally repeat the sound they have heard in the form of a speech output 139.

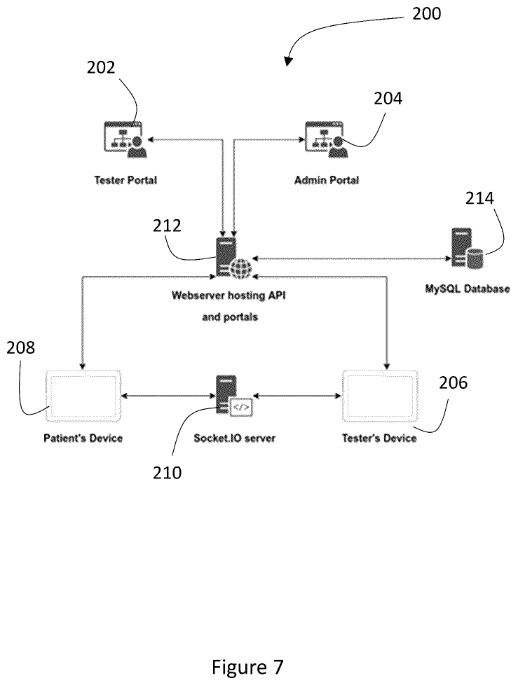

One or more conditions may disrupt proper functioning of one or more of the foregoing processes. One such condition is aphasia. Various tests are known to test aphasia by targeting one or more of the foregoing speech processes that does not correctly function in a person. The aphasia tests, which for the purposes of this disclosure are carried out in Arabic, are tested using a system 200 for performing Arabic aphasia tests on a patient according to one or more embodiments. The system 200 is shown as a block diagram in FIG. 7.

With reference to FIG. 7, the system 200 comprises a testing portal 202, or a tester portal, an admin portal 204, testing device (or tester's device) 206, and a patient device 208. The patient device 208 and the testing device 206 are communicatively linked with each other over a server 210. The server 210 may be a socket.io server. The patient device 208, the testing device 206, the testing portal 202 and the admin portal 204 may be communicatively linked via a webserver 212 hosting API and portals. The webserver 212 may be communicatively linked to a database 214. The API hosted on the webserver 212 may be a REST API. The database 214 may be a MySQL database.

The patient device 208, the testing device 206, the testing portal 202, and the admin portal 204, each include a user interface, a processor, and storage (not shown).

A plurality of tests is stored on the storage of the patient device 208. When executed by the processor, the tests are presented to the patient by the patient device during testing. The patient device 208 is configured to output a stimulus to the patient to present each of the plurality of tests. The stimulus may be an auditory stimulus or a visual stimulus. To output the auditory stimulus, the patient device 208 include a speaker. To output the visual stimulus, the patient device 208 includes a display. A patient may respond to each test by inputting an answer to the user interface of the patient device 208. The user interface may include a tactile input, e.g. a touchscreen, for this purpose. The touch screen may receive written text from the user or options for a user to select. The user interface may also include a microphone for a patient to respond by speaking if the question requires it.

As will be discussed in more detail below, the auditory stimulus may include spoken Arabic. The visual stimulus 209 may include an image of an object or text written in Arabic.

The tester device 206 is configured to output, to the tester, a stimulus that is, in real-time, being output to the patient by the patient device 208. In addition, the tester device 206, is configured to output, to the tester, the answers that are, in real-time, being input by the patient to the patient device 208. In addition, the tester device 206 is configured to receive an input from a tester to control the plurality of tests being presented by the patient device 208. In addition, the tester device 206 is configured to control the plurality of tests being presented by the patient device 208 based on the received input from the tester.

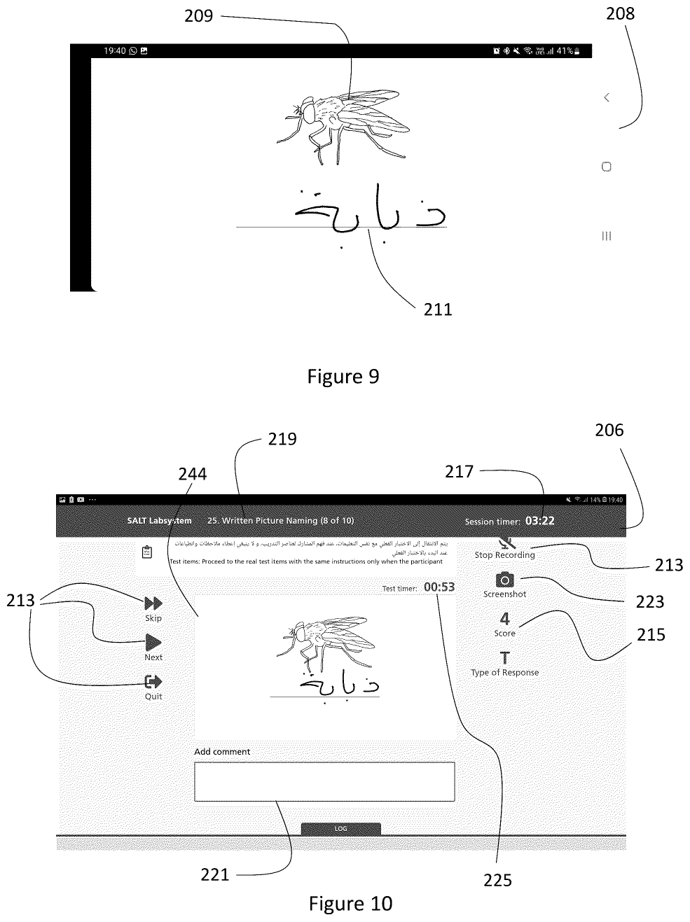

With reference to FIG. 9, the patient device 208 displays a visual stimulus 209 in the form of an insect, e.g. a fly. The patient device 208 receives and displays an answer 211 input by a user in the form of a noun describing the fly.

With reference to FIG. 10, the tester device 206 displays a screenshot 244 of the patient device a plurality of control inputs 213.

The control inputs may be used to control the plurality of tests may comprise sending a controlling action input by the tester, from the tester device 206 to the patient device 208. The controlling action may be selected from a list including skipping a test, jumping a test, interrupting a test, terminating a test, and re-ordering the plurality of tests.

The tester device 206 is also able to receive a mark from a tester using mark input 215. The mark may indicate an answer to a corresponding test is correct. For instance, if the patient has entered an answer, e.g. a written word, or a spoken word, correctly in response to a visual stimulus such as an image of an object, the tester may input a mark indicating that the patient entered the correct answer. The mark may then be sent to the testing portal device 202 together with the corresponding question.

The tester device 206 may also include a session timer 217, a question timer 225, the current test name 219, a comment input 221, and a screenshot request 223 to capture a screenshot of the patient device's display. The comment input and the screenshot request 223 may be configured to receive tactile inputs from the tester.

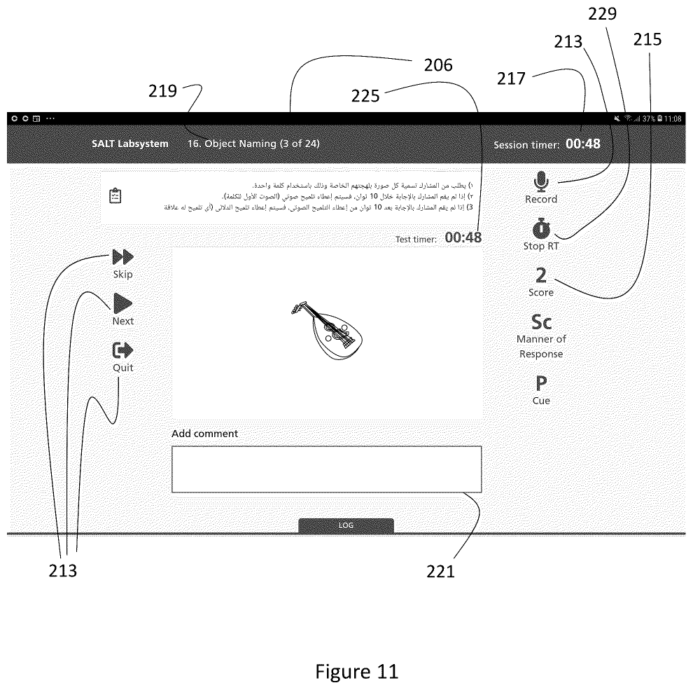

With reference to FIG. 11, a similar screen shot of the tester device 206 is shown as in FIG. 10. In the screen shot of FIG. 11, the tester device 206 is also configured to display a stop response time, RT, input 229. The stop RT input 229 may be an icon for receiving a tactile input from the tester.

The response time may be measured in milliseconds and may correspond to a duration between a first time point and a second time point. The first time point corresponds to a time point at which presentation of a test on the patient device 208 is commenced. The second time point corresponds to a time point at which a patient has finished entering their answer to the patient device 208. As describes herein, the second time point may be detected automatically by the patient device 208 or may be detected manually be input to the tester device 206.

The patient device 208 is configured to measure the response time. However, the response time can only be accurately measured for answers that are entered using a tactile input, e.g. by pressing an icon or entering text on the patient device 208. RT can not be measured accurately enough when the answer involves speech recorded by a microphone of the patient device 208. Therefore, for answers involving speech input answers, the RT is displayed on the tester device 206. When the patient has completed their answer, the tested manually presses the stop RT input 229 to end the RT measurement. The tester device then records the RT and is configured to send the RT to the tester portal device 202 for the tester portal device to include on the report it generates automatically. Where the patient device 208 records the RT for tests including tactile input answers, the patient device is configured to send the RT to the tester portal device 202 for inclusion on the report.

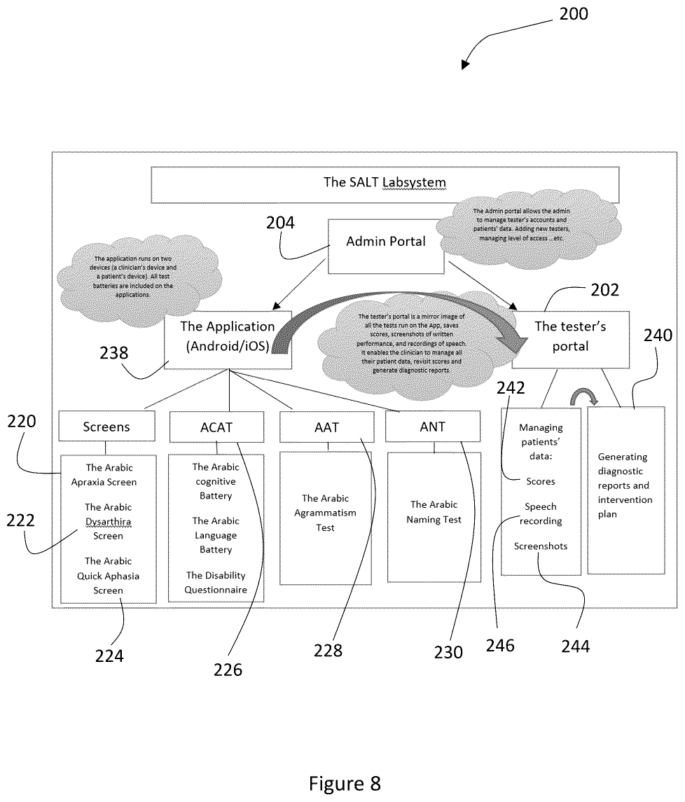

With reference to FIG. 8, the tests to be performed are grouped into categories of test. The tests are stored as an application 238 on the storage of the patient device 208.

The categories of test include an Arabic apraxia screening test 220, Arabic dysarthria screening 222, Arabic quick aphasia screening 224, Arabic comprehensive aphasia testing (ACAT) 226 (which includes various batteries), Arabic agrammatism testing (AAT) 228, and Arabic naming testing (ANT) 230. The batteries included in the ACAT are an Arabic cognitive battery 232, an Arabic language battery 234, and a disability questionnaire 236. Examples of some of the tests are described as follows for illustrative purposes only.

The tester portal 202 is configured to receive, from the tester device 206, via the admin portal 204, a plurality of marks 242, each mark corresponds to an answer input to the patient device 208 indicating that the patient answered the test correctly. The tester portal 202 is configured to generate a report 240 indicating results of the Arabic aphasia tests based on the plurality of tests, the plurality of answers, and the plurality of marks. The report 240 may be a diagnostic report and intervention plan. The tester portal 202 may also display screenshots 244 of the tester device 206 and/or the patient device 208 captured during the language test. The tester portal may also produce a sound recording of speech, i.e. a speech recording 246, captured from the patient device 208 and/or the tester device 206 during testing.

The testing portal may be configured to general an overall aphasia quotient. The overall aphasia quotient may be calculated by first calculating an aphasia quotient using the formula AQ=(S/N)×100. In this formula, AQ is the aphasia quotient, S is a score calculated by counting a number of marks received for a subset of the plurality of tests. The parameter N is a total number of tests within the subset of the plurality of tests.

Next, an overall aphasia quotient may be calculated by calculating a mean value using respective aphasia quotients from a plurality of subsets of the plurality of tests. Each subset of the plurality of tests may correspond to a category of test. For example, the tests making up the ACAT category may form a subset of tests, the tests making up the AAT subset may form another subset of tests, and so forth. The report generated by the tester portal 202 may include the overall aphasia quotient and the aphasia quotient for each category of test. In this way, the tester is able to manually diagnose a subtype of aphasia using the overall aphasia quotient and/or the aphasia quotient for each category of test.

The subtypes of aphasia may be selected from a list including anomic aphasia, conduction aphasia, transcortical motor aphasia, Wernicke's aphasia, transcortical sensory aphasia, Broca's aphasia, isolation aphasia, and global aphasia. The classification of the aphasia subtype depends on the calculation of AQ and patterns of performance on the following ACAT subtests: object naming, verbal fluency, spoken word comprehension, spoken sentence comprehension, and word repetition.

In addition, the testing portal device 202 may be configured to classify automatically an aphasia subtype by comparing aphasia quotient of a respective category of test with a respective threshold. For example, a threshold may be set for each category of test, such as 80%. If a patient scores below 80% in the “object naming” subtest from the ACAT test, the repetition subtests, comprehension subtests and fluency subtests this may indicate a particular subtype (i.e. Global Aphasia). If another patient scores below 80% in in more than one subtest of the ACAT, this may indicate another subtype of aphasia. In this way, the testing portal device 202 may be configured to classify the aphasia subtype depending on whether the aphasia quotients for one or more of the relevant subtests is below the respective threshold. The report may include the automatically classified aphasia subtype.

Next, an example of a plurality of tests within a category of test is provide, specifically for the ACAT 226 test category. This example is for illustrative purposes only.

Table 1 below shows the individual tests that may be performed under the ACAT 226.

TABLE 1
Tests to be performed under ACAT 226

| | Sub- | | Number | Running | | Scoresheet |
|---|---|---|---|---|---|---|
| Category | Category | Test | of Items | Time | | Page # |
| Cognitive | | 1) Line Bisection | 3 | 1 | minute | 9 |
| Screen | | 2) Semantic Memory | 10 | 2 | minutes | 17 |
| | | 3) Word Fluency | 2 | 3 | minutes | 18 |
| | | 4) Visual Recognition Memory | 10 | 1 | minute | 19 |
| | | 5) Gesture Object Use | 6 | 1 | minute | 20 |
| | | 6) Arithmetic | 6 | 2 | minutes | 21 |

In Table 1, the tests in relation to the cognitive screen category of tests are shown. There are other categories of tests within the ACAT, e.g. a disability questionnaire and the Arabic language battery.

An example of the tests is provided be illustrating 1) line bisection.

The purpose of line bisection is to detect visual neglect/field defects through a line bisection task.

The components of the test include horizontal lines configured to appear in random positions on the page/screen. There may be 3 horizontal lines as practice items and 3 horizontal lines as test items.

The tester will first administer the practice items on a practice page/screen on the patient device 208 and explain the instructions clearly. Feedback should be given to the patient after each trial on the practice page/screen.

The tester will ask the participant via the tester device 206 to cut each horizontal line in half, by drawing a vertical line down the centre of each horizontal line on the patient device 208.

The tester should proceed to the real test only after the participant demonstrates understanding of the presented task on the practice page/screen. The tester can proceed to the real test by using one or more of the control inputs 213 on the tester device 206.

Once the real test begins, feedback should not be given to the patient via the patient device 208.

Test instructions for the practice items include asking the patient to divide each line in half on the screen of the tester device 208, by drawing a vertical line down the centre of each horizontal line, and providing feedback when the demonstration is correct/incorrect.

Test instructions for real test items include proceeding to the real test items with the same instructions as the practice items only when the participant had demonstrated understanding of the test with the practice items. Any feedback functions on the tester device 206 may be disabled to prevent any feedback being given to the patient on the patient device 208 during the test.

The tests may be marked as follows. One mark may be entered by the tester on the tester device 206 for each correct bisection entered by the patient on the patient device 208. The tester may enter on the tester device 206 the total number of marks for a respective number of lines that were correctly bisected. If the patient failed to enter at least two lines correctly, the tester may discontinue the test using the corresponding control input 213. It should be noted that practice items are not marked by the tester.

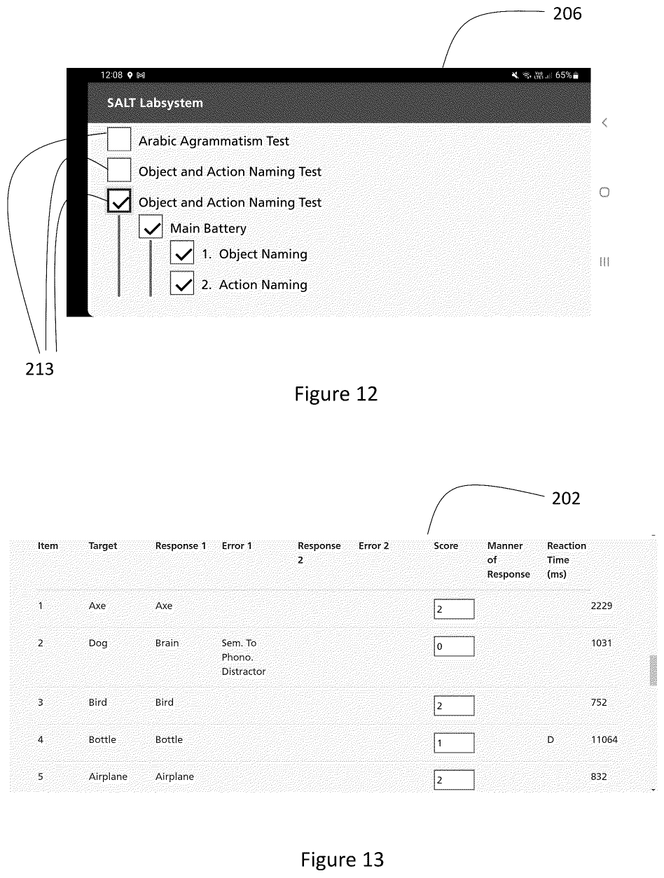

With reference to FIG. 12, a screen shot from the tester device 206 shows a test selector. The test selector is effectively another control input 213 where a tester may select tests to be carried out by the patient on the patient device 208. It can be seen that the control inputs 213 are provided as tick boxes, where a tester may select a test to be carried out by the patient on the patient device 208 by entering a tick in the corresponding tick box. Any blank tick boxes mean that the test has not be selected so will not be presented to the patient on the patient device 208.

With reference to FIG. 13, a screen shot of the tester portal device 202 is shown. The screen shot includes tests answered by the patient on the tester device 208, together with the reaction time, the response, and a score entered by the tester from the tester device 206.

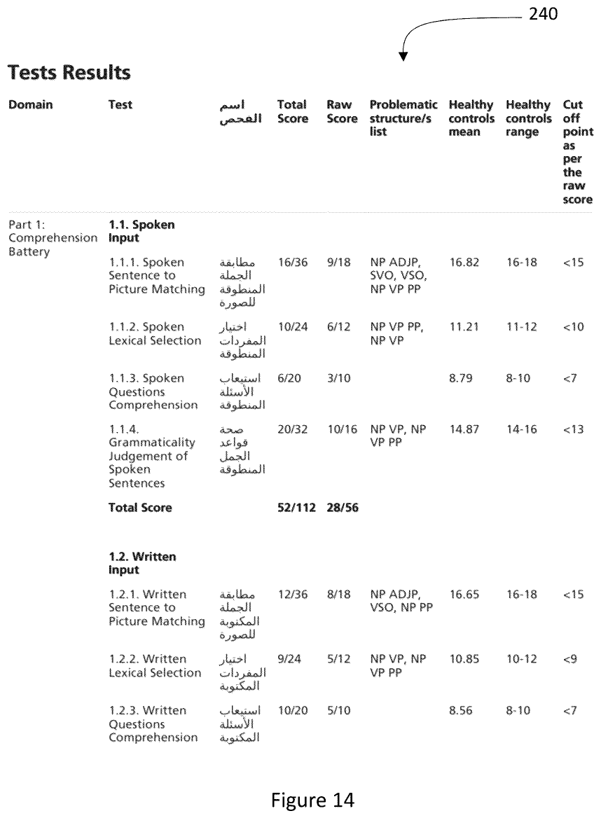

With reference to FIG. 14, the tester portal device 202 may generate a report 240 indicating results of the Arabic aphasia tests based on the plurality of tests, the plurality of answers, and the plurality of marks. The report 240 may relate to the AAT. The report 240 may include a category of the tests presented to the patient on the patient device 208, the individual tests within that category, the answer, a total score, a raw score, a list of problematic structures, healthy controls mean and range which may be based on a sample of previous patients' answers, and a threshold associated with the category of test, which may be called a cut off point.

The foregoing system 200 may be described in terms of a method of operation. The method may be defined by a set of instructions stored on a transitory computer-readable medium. When the instructions are executed by one or more processors, the one or more processors may be configured to perform the methods. The methods may be summarised as follows.

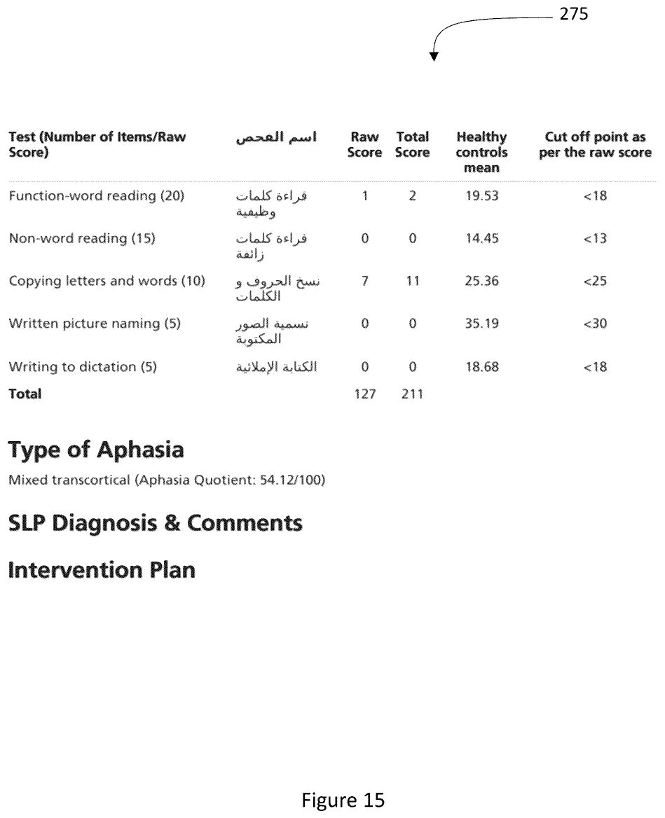

With reference to FIG. 15, a further report 275 may be generated by the tester portal device 202 in relation to the ACAT. The report may include the individual tests within that category, the answer, a total score, a raw score, healthy controls mean which may be based on a sample of previous patients' answers, and a threshold associated with the category of test, which may be called a cut off point. The further report 275 may also include the aphasia quotient and the aphasia subtype diagnosis.

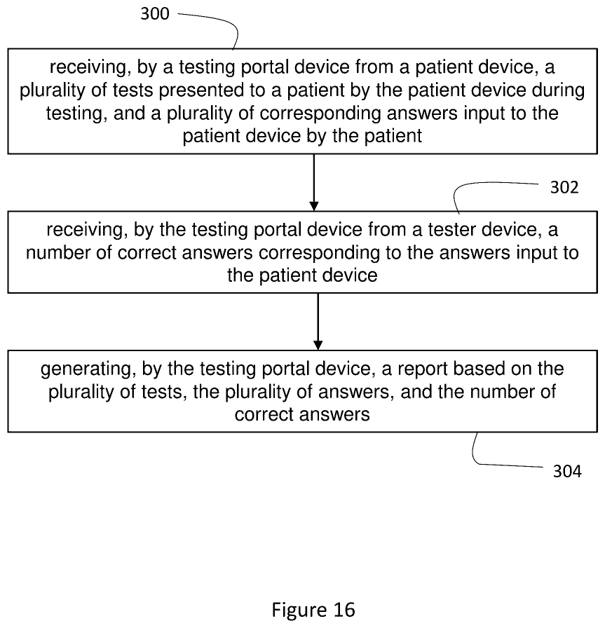

With reference to FIG. 16, there is provided a computer-implemented method, according to one or more embodiments, of performing Arabic aphasia tests on a patient. The method comprises: receiving 300, by a testing portal device 202 from a patient device, a plurality of tests presented to a patient by the patient device 208 during testing, and a plurality of corresponding answers input to the patient device 208 by the patient; receiving 302, by the testing portal device 202 from a tester device 206, a number of correct answers corresponding to the answers input to the patient device 208; and generating 304, by the testing portal device 202, a report 240 based on the plurality of tests, the plurality of answers, and the number of correct answers.

Whilst the following embodiments provide specific illustrative examples, those illustrative examples should not be taken as limiting, and the scope of protection is defined by the claims. Features from specific embodiments may be used in combination with features from other embodiments without extending the subject-matter beyond the content of the present disclosure.

1. A system for performing Arabic aphasia tests on a patient, the system comprising:
a testing portal device, the testing portal device in communication with a patient device and a tester device, the testing portal device configured to:
receive, from the patient device, a plurality of tests presented to the patient by the patient device during testing, and a plurality of corresponding answers input to the patient device by the patient;
receive, from the tester device, a plurality of marks, each mark corresponding to an answer input to the patient device indicating that the patient answered the test correctly; and
generate a report indicating results of the Arabic aphasia tests based on the plurality of tests, the plurality of answers, and the plurality of marks.

2. The system of claim 1, wherein the testing portal device is configured to generate an overall aphasia quotient by:
calculating an aphasia quotient using AQ=(S/N)×100, where AQ is the aphasia quotient, S is a score calculated by counting a number of marks received for a subset of the plurality of tests, and N is a total number of tests within the subset of the plurality of tests; and
calculating the overall aphasia quotient by calculating a mean value using respective aphasia quotients from a plurality of subsets of the plurality of tests, wherein each subset of the plurality of tests corresponds to a category of test,
wherein the report includes the overall aphasia quotient and the aphasia quotient for each category of test.

3. The system of claim 2, wherein the testing portal device is configured to classify automatically an aphasia subtype by comparing each aphasia quotient of a respective category of test with a respective threshold, and to classify the aphasia subtype depending on whether one or more of the aphasia quotients is below the respective threshold, wherein the report includes the automatically classified aphasia subtype.

4. The system of claim 3, wherein the aphasia subtype is selected from a list including anomic aphasia, conduction aphasia, transcortical motor aphasia, Wernicke's aphasia, transcortical sensory aphasia, Broca's aphasia, isolation aphasia, and global aphasia.

5. The system of claim 2, wherein the category of tests includes Arabic apraxia screening, Arabic dysarthria screening, Arabic quick aphasia screening, Arabic comprehensive aphasia testing, Arabic naming testing, and Arabic agrammatism testing.

6. The system of claim 1, further comprising the patient device, wherein the patient device is in communication with the tester device and is configured to:
output a stimulus to the patient to present each of the plurality of tests; and
receive an input from the patient, wherein the input includes a response to the respective test.

7. The system of claim 6, wherein the stimulus is a stimulus selected from a list including an auditory stimulus and a visual stimulus.

8. The system of claim 7, wherein, when the stimulus is an auditory stimulus, the auditory stimulus includes an auditory stimulus comprises spoken Arabic.

9. The system of claim 7, wherein, when the stimulus is a visual stimulus, the visual stimulus includes a visual stimulus selected from a list including Arabic text and an image.

10. The system of claim 6, wherein the input from the patient is an input selected from a list including a tactile input, and a phonetic input.

11. The system of claim 1, wherein the patient device is configured to measure a response time, wherein the response time is a time between a first time point when a test is displayed and a second time point when a patient has finished inputted the corresponding answer.

12. The system of claim 1, further comprising the tester device, wherein the tester device is in communication with the patient device and is configured to:
output, to the tester, a test of the plurality of tests that is, in real-time, being output to the patient by the patient device;
output, to the tester, the answers that are, in real-time, being input by the patient to the patient device;
receive an input from a tester to control the plurality of tests being presented by the patient device; and
controlling the plurality of tests being presented by the patient device based on the received input from the tester.

13. The system of claim 12, wherein the controlling the plurality of tests being presented by the patient device comprising a controlling action selected from a list including skipping a test, jumping a test, interrupting a test, terminating a test, and re-ordering the plurality of tests.

14. The system of claim 12, wherein the tester device is configured to:
receive a mark from the tester, the mark indicating that an answer to a corresponding test is correct; and
send the mark to the testing portal device together with the corresponding question.

15. The system of claim 12, wherein the tester device is configured to display a response time, wherein the response time includes a timer starting at a first time point, the first time point corresponding to a time when the test starts to be presented on the patient device, and wherein the tester device is configured to receive an input from the tester to stop the timer.

16. A computer-implemented method of performing Arabic aphasia tests on a patient, the method comprising:
receiving, by a testing portal device from a patient device, a plurality of tests presented to a patient by the patient device during testing, and a plurality of corresponding answers input to the patient device by the patient;
receiving, by the testing portal device from a tester device, a number of correct answers corresponding to the answers input to the patient device; and
generating, by the testing portal device, a report based on the plurality of tests, the plurality of answers, and the number of correct answers.

17. The method of claim 14, wherein the generating the report comprises generating an overall aphasia quotient by:
calculating, by the testing portal device, an aphasia quotient using a formula AQ=(S/N)×100, where AQ is the aphasia quotient, S is a score calculated by counting the number of marks awarded for a subset of the plurality of tests, and N is a total number of tests within the subset of the plurality of tests; and
calculating, by the testing portal device, the overall aphasia quotient by calculating a mean value using respective aphasia quotients from a plurality of subsets of the plurality of tests, wherein each subset corresponds to a category of test,
wherein the report includes the overall aphasia quotient and the aphasia quotient for each category of test.

18. The method of claim 15, further comprising classifying automatically an aphasia subtype by comparing each aphasia quotient of a respective category of test with a respective threshold, and classifying the aphasia subtype depending on whether one or more of the aphasia quotients is below the respective threshold, wherein the report includes the automatically classified aphasia subtype.

19. The method of claim 14, further comprising :
outputting, by the patient device, a stimulus to the patient to present each of the plurality of tests; and
receiving, by the patient device, an input from the patient, wherein the input includes a response to the respective test.

20. A non-transitory computer-readable medium including instructions stored thereon that when executed by a processor, cause the processor to perform the method of claim 16.
